(19) United States
(12) Patent Application Publication
(10) Pub. No.: US 2005/0158331 A1
(43) Pub. Date: Jul. 21, 2005

(54) Autogenous vaccine derived from one's own hypothalamus of the brain & kidney's via a process known as liquefaction

(76) Inventor: Randall Aaron Morton, Laguna Niguel, CA (US)

Correspondence Address: STETINA BRUNDA GARRED & BRUCKER, 75 ENTERPRISE, SUITE 250, ALISO VIEJO, CA, 92656, US

(21) Appl. No.: 11/079,156

(22) Filed: Mar. 14, 2005

Related U.S. Application Data
(63) Continuation of application No. 09/840,677, filed on Apr. 23, 2001, now abandoned.

(51) Int. Cl. A61K 39/00

(52) U.S. Cl. 424/184.1

(57) ABSTRACT

By extrapolation of tissue from the person affected, and then using the process of liquefaction, and from there creating an autogenous vaccine and injecting that vaccine 1 cc to the affected area would not only cure heart disease, strokes, renal failure, Chron's Disease, asthma, sugar diabetes and as a cure for paralyses would not pose rejection since it would be coming from one's own tissues.

This invention involves the extrapolation of tissue from the hypothalamus of the brain and tissue from the medulla of the brain stem and tissue from the renal medulla of the kidney (which is located in the right kidney) and tissue from both the cortex of the brain and kidney from the person involved. Then, by using a process called liquefaction (which is a conversion of tissue to a fluid or semi fluid state), create an autogenous vaccine for the cure, treatment and prevention of heart disease, strokes, renal failure, Chron's Disease, asthma, sugar diabetes and as a cure for paralyses. The vaccine would be administered through injection of 1 cc to the affected area (s).

Treatment of hear disease, strokes, renal failure, Chron's Disease, asthma, sugar diabetes and paralysis.

Treatment of the above mentioned diseases have been dismal at best. In the case of heart disease, the common forms of treatment are angioplasty, which relieves blockage to the heart. This, however, must be repeated and is not a cure. Also, heart bypass surgery has been tried and that too had to be repeated and again has proven to be no cure for heart disease. Heart transplants have been tried, often times the body has rejected these hearts and again, this is not a cure. Each treatment is followed up by a regimen of drugs that those afflicted take for the duration of their lives only to experience severe side effects.

When a person has a stroke and is fortunate enough to get treatment with a clot buster known as TPA within 3 hours after the onset of a stroke, studies have shown that a stroke could be reversed, and that many doctors are hesitant to use TPA.

Those that suffer from renal failure are candidates for the current treatments which are kidney transplants or dialysis 3 to 4 times per week. Waiting lists for kidneys are quite long, not to mention possible rejection and the unwelcome side effects of antirejection medications. Dialysis offers only a short term solution as well as unwelcome side effects.

Asthma patients can only rely on drugs and inhalation therapy to get some relief. Again, this treatment does not represent a cure for asthma. There are also drug interaction side effects for many.

Sugar diabetes afflicts millions of people. Currently, insulin, diet, or if one is borderline, medications are used. Again, these treatments do not cure sugar diabetes.

1. A method for creating an autogenous vaccine useful in the treatment of various medical conditions by the extrapolation of tissue from the hypothalamus and cortex of the brain, the medulla of the brain stem, the cortex of the kidney, and the renal medulla of the person involved and then converting the extrapolated tissue into a fluid or semi-fluid state.

2-8. (canceled)

9. A vaccine useful in treating various medical conditions derived from the hypothalamus and cortex of the brain, medulla of the brain stem, cortex of the kidney, and renal medulla tissue of the intended recipient wherein such tissue has been converted into a fluid or semi-fluid state.

10. A method for treating a medical condition via the onetime injection of 1 cc of the vaccine in claim 9 directly into the site wherefrom the medical condition arises.

11. The method of claim 10 where the medical condition is heart disease and the vaccine is injected directly into the heart muscle.

12. The method of claim 10 where the medical condition is a stroke and the vaccine is injected directly into the pituitary gland.

13. The method of claim 10 where the medical condition is renal failure and the vaccine is injected directly into the common hepatic artery.

14. The method of claim 10 where the medical condition is Chron's Disease and the vaccine is injected directly into the stomach.

15. The method of claim 10 where the medical condition is asthma and the vaccine is injected directly into the chest cavity.

16. The method of claim 10 where the medical condition is sugar diabetes and the vaccine is injected directly into the Iles of Langrahan.

17. The method of claim 10 where the medical condition is paralysis and the vaccine is injected directly into the spinal sack.
